(19) United States
(12) Patent Application Publication
(10) Pub. No.: US 2016/0177390 A1
(43) Pub. Date: Jun. 23, 2016

(54) GENETIC AND IMAGE BIOMARKETS ASSOCIATED WITH DECLINE IN COGNITIVE MEASURES AND BRAIN GLUCOSE METABOLISM IN POPULATIONS WITH ALZHEIMER'S DISEASE OR THOSE SUSCEPTIBLE TO DEVELOPING ALZHEIMER'S DISEASE

(71) Applicant: BIOGEN INTERNATIONAL NEUROSCIENCE GMBH, Zug (CH)

(72) Inventors: Sheng Feng, Lexington, MA (US); Jeff Sevigny, Lexington, MA (US); Ajay Verma, Bedford, MA (US); Donald Bennett, N. Easton, MA (US); Yen Ying Lim, Providence, RI (US); Paul Thomas Maruff, Victoria (AU)

(21) Appl. No.: 14/904,388

(22) PCT Filed: Jul. 9, 2014
(86) PCT No.: PCT/US2014/045994

§ 371 Date: Jan. 11, 2016

Related U.S. Application Data
(60) Provisional application No. 61/845,925, filed on Jul. 12, 2013.

(51) Int. Cl. C12Q 1/68; C07K 16/18

(52) U.S. Cl. CPC C12Q 1/6883; C07K 16/18; C12Q 2600/118; C12Q 2600/156; C07K 2317/565; C07K 2317/56

(57) ABSTRACT

The present disclosure is based on the identification of biomarkers of combined genetic variants and imaging measurements, in predicting faster decline in cognitive measures and brain glucose metabolism in populations with Alzheimer's disease or those susceptible to developing Alzheimer's disease. The present disclosure provides a method of treating a patient with Alzheimer's disease (AD) or a subject susceptible to developing AD, comprising: (a) assaying a sample obtained from an early-stage AD patient or a subject susceptible to developing AD for the presence of a brain-derived neurotrophic factor (BDNF) gene mutation and/or a protein tyrosine phosphatase receptor-type, Z polypeptide 1 (Ptprz1) gene mutation; (b) determining whether the patient or subject is positive for brain amyloid-beta (Aβ), wherein the presence of brain Aβ in combination with the BDNF gene and/or Ptprz1 gene mutation correlates with a prediction of rapid cognitive decline; and (c) treating the patient or subject with early and aggressive therapy appropriate to treat AD with rapid cognitive decline.

This application claims priority to U.S. Provisional Patent Application No. 61/845,925 filed Jul. 12, 2013, the contents of which are incorporated herein by reference.

The content of the electronically submitted sequence listing in ASCII text file (Name: 21594160000SequenceListing_ascii.txt; Size: 3.82 KB; and Date of Creation: Jul. 12, 2013) filed with the application is incorporated herein by reference in its entirety.

### BACKGROUND

The present disclosure is based in part on the identification of biomarkers of combined genetic variants and imaging measurements. These biomarkers are useful, e.g., in predicting faster decline in cognitive measures and brain glucose metabolism in a patient with Alzheimer's disease or a subject susceptible to developing Alzheimer's disease.

Alzheimer's disease (AD) is a progressive, neurodegenerative disorder characterized by amyloid deposition in the cerebral neuropil and vasculature. These amyloid deposits comprise predominantly fragments and full-length (40 or 42 residue) forms of the amyloid β-protein (Aβ) organized into fibrillar assemblies (Kirkitadze et al., Journal of Neuroscience Research 69:567-577 (2002)). Alzheimer's disease (AD) accounts for approximately two-thirds of late-life dementias, afflicting an estimated 8% of people age 65 years and older (Ritchie K. and Kildea D., Lancet 346:931-934 (1995)).

Genetic biomarkers predicting cognitive decline in Alzheimer's disease, especially in early disease stages, are important for understanding disease pathology and designing efficient clinical trials. For example, statistical analyses of the Australian Imaging, Biomarker and Lifestyle (AIBL) Flagship Study of Aging data revealed that a mutation, Val66Met at rs6265, in the brain-derived neurotrophic factor (BDNF) gene is strongly associated with faster cognitive decline with the presence of brain amyloid in the normal-to-early Alzheimer's disease population.

The use of positron emission tomography (PET) imaging with probes that bind specifically to β-amyloid and tau aggregates has received increased attention recently because this technique can provide an earlier diagnosis of AD. To be clinically diagnosed, AD must reach the dementia stage, in which cognitive and non-cognitive symptoms significantly alter activities of daily living. However, disease symptoms may begin to appear years before initial clinical manifestations. Thus, the new AD diagnostic criteria suggest that the diagnosis of “prodromal AD” (also called the AD predementia stage) or mild cognitive impairment “MCI” due to AD pathology should rely on in-vivo biomarkers of amyloid pathology. For example, PET imaging that uses ligands of amyloid plaques and degenerative neurofibrillary tangles, such as pittsburgh compound B positron (PiB) (N-methyl-[¹¹C]2-(4′-methylaminophenyl)-6-hydroxybenzothiazole), [¹⁸F]-labelled amyloid ligands, such as [¹⁸F]-fluorodeoxyglucose ([¹⁸F]-FDG), and [¹⁸F]-AV-45 (florbetapir) (Camus et al., Eur J Nucl Med Mol Imaging. 39(4): 621-631 (2012)).

Although AD is a progressive neurodegenerative condition, there is great intra- and inter-individual variability in rates of cognitive decline. (Teri et al., J Gerontol A Biol Sci Med Sci 50A (1):M49-M55 (1995)). So far, little data exist to explain such variability. Accordingly, there is a need to develop methods and analytical approaches combining the identification of genetic and image biomarkers predicting cognitive decline in populations with AD or those susceptible to developing AD.

### BRIEF SUMMARY

The present disclosure provides a method of treating a patient with Alzheimer's disease (AD) or a subject susceptible to developing AD, comprising: (a) assaying a sample obtained from an early-stage AD patient or a subject susceptible to developing AD for the presence of a brain-derived neurotrophic factor (BDNF) gene mutation and/or a protein tyrosine phosphatase receptor-type, Z polypeptide 1 (Ptprz1) gene mutation; (b) determining whether the patient or subject is positive for brain amyloid-beta (Aβ), wherein the presence of brain Aβ in combination with the BDNF gene and/or Ptprz1 gene mutation correlates with a prediction of rapid cognitive decline; and (c) treating the patient or subject with early and aggressive therapy appropriate to treat AD with rapid cognitive decline.

Also disclosed is a method of treating a patient with AD or a subject susceptible to

developing AD, comprising: (a) assaying a sample obtained from an early-stage AD patient or a subject susceptible to developing AD for the presence of a BDNF gene and/or Ptprz1 gene mutation; (b) determining whether the patient or subject is positive for brain Aβ, wherein the presence of brain Aβ in combination with the BDNF gene and/or Ptprz1 gene mutation correlates with a prediction of rapid cognitive decline; and (c) instructing a healthcare provider to administer early and aggressive therapy appropriate to treat AD with rapid cognitive decline.

Further disclosed is a method of treating a patient with AD or a subject susceptible to developing AD, comprising: (a) obtaining a sample from an early-stage AD patient or a subject susceptible to developing AD, and submitting the sample for determination of the presence of a BDNF gene and/or Ptprz1 gene mutation; (b) ordering a test to determine whether the patient or subject is positive for brain Aβ, wherein the presence of brain Aβ in combination with the BDNF gene and/or Ptprz1 gene mutation correlates with a prediction of rapid cognitive decline; and (c) treating the patient or subject with early and aggressive therapy appropriate to treat AD with rapid cognitive decline.

Also disclosed is a method of treating a patient with AD or a subject susceptible to developing AD, comprising administering to the patient or subject an anti-Aβ antibody, or antigen-binding fragment thereof, a cholinesterase inhibitor, an N-methyl-D-aspartate receptor antagonist, or any combination thereof, wherein the patient has (a) at least one mutation in a BDNF gene and/or Ptprz1 gene and (b) brain Aβ.

Also disclosed is a method of prognosing a patient with AD or a subject susceptible to developing AD, comprising: (a) assaying a sample obtained from an early-stage AD patient or a subject susceptible to developing AD for the presence of a BDNF gene and/or Ptprz1 gene mutation; and (b) determining whether the patient or subject is positive for brain Aβ; wherein the presence of brain Aβ in combination with the BDNF gene and/or Ptprz1 gene mutation correlates with a prediction of rapid cognitive decline, and indicates a need for rapid, aggressive AD treatment.

Also disclosed is a method of predicting the rate of cognitive decline expected in a patient with AD or a subject susceptible to developing AD, comprising: (a) assaying a sample obtained from an early-stage AD patient or a subject susceptible to developing AD for the presence of a BDNF gene and/or Ptprz1 gene mutation; and (b) determining whether the patient or subject is positive for brain Aβ; wherein the presence of brain Aβ in combination with the BDNF gene and/or Ptprz1 gene mutation correlates with a prediction of rapid cognitive decline, and indicates a need for rapid, aggressive AD treatment.

Further disclosed is a method of predicting the rate of cognitive decline expected in a patient with AD or a subject susceptible to developing AD, comprising: (a) obtaining a sample from an early-stage AD patient or a subject susceptible to developing AD, and submitting the sample for determination of the presence of a BDNF gene and/or Ptprz1 gene mutation; and (b) ordering a test to determine whether the patient or subject is positive for brain Aβ; wherein the presence of brain Aβ in combination with the BDNF gene and/or Ptprz1 gene mutation correlates with a prediction of rapid cognitive decline, and indicates a need for rapid, aggressive AD treatment.

Certain embodiments include the method as described herein, wherein the presence of brain Aβ in combination with a BDNF gene mutation further correlates with a prediction of decline in brain glucose metabolism, as measured by [¹⁸F]-fluorodeoxyglucose positron emission tomography (FDG-PET).

In some embodiments, brain Aβ is measured by pittsburgh compound B positron emission tomography PiB-PET or [¹⁸F]-AV-45 (florbetapir)-PET.

In certain embodiments, the sample from an early-stage AD patient or a subject susceptible to developing AD comprises fresh, frozen, or preserved tissue, a biopsy, an aspirate, blood or any blood constituent, a bodily fluid, cells, or any combination thereof.

In some embodiments, the sample is assayed for the presence of the BDNF gene or Ptprz1 gene mutation using a nucleic acid hybridization assay, a nucleic acid polymerization assay, a sequencing assay, or a combination thereof.

In some embodiments, the assay comprises the use of a gene chip array.

In certain embodiments, the assay comprises a TaqMan assay, a flap endonuclease assay, genomic DNA sequencing.

In some embodiments, the presence of the BDNF gene or Ptprz1 gene mutation is determined using a nucleic acid probe specific for the mutation.

In some embodiments, comprises a single nucleotide polymorphism (SNP).

In some embodiments, the BDNF gene or Ptprz1 gene mutation comprises two or more SNPs.

In some embodiments, the BDNF gene mutation comprises at least one copy of Val66Met (A/G) at rs6265.

In some embodiments, the BDNF gene mutation comprises two copies of Val66Met (A/G) at rs6265.

In some embodiments, a patient positive for both brain Aβ and at least one copy of the Val66Met mutation is predicted to have a faster 36 month cognitive decline than a patient negative for either brain Aβ or a Val66Met mutation.

In some embodiments, the Ptprz1 gene mutation comprises at least one copy of “T” allele at rs6946211.

In some embodiments, the rate of cognitive decline can be measured by a mini-mental state examination, the clinical dementia rating scale, the Boston name test, a logical memory test, a delayed recall test, or any combination thereof.

In some embodiments, a patient positive for both brain Aβ and at least one copy of the Val66Met mutation is predicted to have a faster decline in brain glucose metabolism than a patient negative for either brain Aβ or a Val66Met mutation.

In some embodiments, the therapy comprises administration of an anti-Aβ antibody, or antigen-binding fragment thereof, a cholinesterase inhibitor, an N-methyl-D-aspartate receptor antagonist, or any combination thereof.

In some embodiments, the antibody or fragment thereof is can bind a beta-amyloid plaque, a cerebrovascular amyloid, a diffuse Abeta deposit, a neurofibrillary tangle, or an Abeta protein aggregate; wherein the antibody or its encoding cDNA is derived from B-cells or memory B-cells obtained from a human patient who is symptom-free but affected with or at risk of developing a disorder, or a human patient with an unusually stable disease course, and wherein the antibody has been identified by binding to a specimen of pathologically altered cells or tissue of predetermined clinical characteristics.

In some embodiments, the antibody or fragment thereof comprises a VH and a VL, wherein the VH comprises VHCDR1, VHCDR2, and VHCDR3 amino acid sequences of SEQ ID NOs: 3, 4, and 5, respectively, and the VL, comprises VLCDR1, VLCDR2, and VLCDR3 amino acid sequences of SEQ ID NOs: 6, 7, and 8, respectively.

In some embodiments, the antibody or fragment thereof comprises a VH and a VL, wherein the VH comprises SEQ ID NO: 1 and the VL comprises SEQ ID NO: 2.

### BRIEF DESCRIPTION OF THE DRAWINGS/FIGURES

FIGS. 1A to 1F show multiple cognitive measures in patients of the early Alzheimer's disease population. 36 month cognitive decline is measured by six clinical measurements: (A) Mini-Mental State Examination (MMSE), (B) Clinical Dementia Rating Scale (CDR), (C) Boston Name Test (BNTT), (D) Logical Memory Delayed Recall (LDEL), (E) 30 Minute Delay Total (AVDEL30MIN), and (F) Neuropsychological Battery Test: digitscore=Total Correct. Individuals with at least one copy of “A” allele at rs6265 in the BDNF gene and positive Aβ scans are coded as “11;” double-negative are “00;” positive Aβ scan but no mutation are “01;” and negative Aβ scan with mutation are “10.” m=mean; std=standard deviation; General linear model ANOVA analysis applied.

FIGS. 2A to 2C show 36 month cognitive decline measured by MMSE in patients of the early Alzheimer's disease population. Individuals with (A) the BDNF gene rs11030104 SNP (“BDNF1”), (B) the BDNF gene rs12273363 SNP (“BDNF2”), or (C) the BDNF gene rs908867 SNP (“BDNF3”), and positive Aβ scans are coded as “11;” double-negative are “00;” positive Aβ scan but no mutation are “01;” and negative Aβ scan with mutation are “10.” m=mean; std=standard deviation; General linear model ANOVA analysis applied.

FIG. 3 shows 36 month cognitive decline measured by MMSE in patients of the early Alzheimer's disease population. Individuals with at least one copy of “T” allele at rs6946211 in the Ptprz1 gene and positive Aβ scans are coded as “11;” double-negative are “00;” positive Aβ scan but no mutation are “01;” and negative Aβ scan with mutation are “10.” m=mean; std=standard deviation; General linear model ANOVA analysis applied.

FIG. 4 shows a measure of brain glucose metabolism in patients of the early Alzheimer's disease population. Individuals with at least one copy of “A” allele at rs6265 in the BDNF gene) are coded as “Val/Met;” double-negative are “Val/Val;” and double positive (two copies of “A” allele at rs6265 in the BDNF gene) are coded “Met/Met.” Aβ-negative=negative Aβ scan and Aβ-positive=positive Aβ scan; m=mean; std=standard deviation; FDG=[¹⁸F]-fluorodeoxyglucose. Both 1 year and 2 year change was evaluated; General linear model ANOVA analysis applied.

### DETAILED DESCRIPTION OF THE INVENTION

### I. Definitions

It must be noted that, as used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described herein with the language “comprising,” otherwise analogous embodiments described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety. Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “neurodegenerative disease” includes but is not limited to Alzheimer's Disease, mild cognitive impairment, fronto-temporal dementia, Lewy-body disease, Parkinson's disease, Pick's disease, Binswanger's disease; congophilic amyloid angiopathy, cerebral amyloid angiopathy, Down's syndrome, multi-infarct dementia, Huntington's Disease, Creutzfeldt-Jakob Disease, AIDS dementia complex, depression, anxiety disorder, phobia, Bell's Palsy, epilepsy, encephalitis, multiple sclerosis; neuromuscular disorders, neurooncological disorders, brain tumors, neurovascular disorders including stroke, neuroimmunological disorders, neurootological disease, neurotrauma including spinal cord injury, pain including neuropathic pain, pediatric neurological and neuropsychiatric disorders, sleep disorders, Tourette syndrome, mild cognitive impairment, vascular dementia, multi-infarct dementia, cystic fibrosis, Gaucher's disease other movement disorders and disease of the central nervous system (CNS) in general. Unless stated otherwise, the terms “neurodegenerative,” “neurological” or “neuropsychiatric” are used interchangeably herein.

Unless stated otherwise, the terms “disorder,” “disease” and “illness” are used interchangeably herein.

Unless stated otherwise, the terms “Aβ,” “Abeta” and “beta-amyloid” are used interchangeably herein. Unless specifically indicated, the terms refer to any form of beta-amyloid, e.g., Aβ-39, Aβ-40, Aβ-41, and Aβ-42.

As used herein, the terms “binding molecule” or “antigen binding molecule” refers in its broadest sense to a molecule that specifically binds an antigenic determinant. Non-limiting examples of antigen binding molecules are antibodies and fragments thereof that retain antigen-specific binding, as well as other non-antibody molecules that bind to an antigen of interest, e.g., Aβ, including but not limited to hormones, receptors, ligands, major histocompatibility complex (MHC) molecules, chaperones such as heat shock proteins (HSPs), as well as cell-cell adhesion molecules such as members of the cadherin, intergrin, C-type lectin, and immunoglobulin (Ig) superfamilies. Thus, for the sake of clarity only and without restricting the scope of the disclosure, most of the following embodiments are discussed with respect to antibodies and antibody-like molecules which represent the binding molecules for the development of therapeutic and diagnostic agents. In another embodiment, a binding molecule disclosed comprises at least one heavy or light chain CDR of an antibody molecule. In another embodiment, a binding molecule disclosed comprises at least two CDRs from one or more antibody molecules. In another embodiment, a binding molecule disclosed comprises at least three CDRs from one or more antibody molecules. In another embodiment, a binding molecule as disclosed comprises at least four CDRs from one or more antibody molecules. In another embodiment, a binding molecule as disclosed comprises at least five CDRs from one or more antibody molecules. In another embodiment, a binding molecule as disclosed comprises at least six CDRs from one or more antibody molecules.

Unless specifically referring to full-sized antibodies such as naturally occurring antibodies, the term “anti-Aβ antibody” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988) Antibodies: A Laboratory Manual (2nd ed.; Cold Spring Harbor Laboratory Press).

As used herein, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the disclosure and, accordingly, are within the scope of the disclosure. All immunoglobulin classes are clearly within the scope of the disclosure. The following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL or VK) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs) within these variable domains, of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule can consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see below).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al. (1983) U.S. Dept. of Health and Human Services, “Sequences of Proteins of Immunological Interest” and by Chothia and Lesk, J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest.” Unless otherwise specified, references to the numbering of specific amino acid residue positions in an anti-Aβ antibody or antigen-binding fragment, variant, or derivative thereof of the present disclosure are according to the Kabat numbering system.

As used herein, the term “chimeric antibody” will be held to mean any antibody wherein the immunoreactive region or site is obtained or derived from a first species and the constant region (which can be intact, partial or modified in accordance with the instant disclosure) is obtained from a second species. For example, the target binding region or site can be from a non-human source (e.g., mouse or primate) and the constant region can be human. Alternatively, a fully human binding region can be combined with a non-human (e.g., mouse) constant region.

As used herein, “human” or “fully human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example, in U.S. Pat. No. 5,939,598 by Kucherlapati et al. “Human” or “fully human” antibodies also include antibodies comprising at least the variable domain of a heavy chain, or at least the variable domains of a heavy chain and a light chain, where the variable domain(s) have the amino acid sequence of human immunoglobulin variable domain(s).

“Human” or “fully human” antibodies also include “human” or “fully human” antibodies, as described herein, that comprise, consist essentially of, or consist of, variants (including derivatives) of antibody molecules (e.g., the VH regions and/or VL regions) described herein, which antibodies or fragments thereof immunospecifically bind to an Aβ polypeptide or fragment or variant thereof. Standard techniques known to those of skill in the art can be used to introduce mutations in the nucleotide sequence encoding a human anti-Aβ antibody, including, but not limited to, site-directed mutagenesis and PCR-mediated mutagenesis which result in amino acid substitutions. In certain embodiments the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

In one aspect, the antibody of the disclosure is a human monoclonal antibody as derived from human B cells. Optionally, the framework region of the human antibody is aligned and adopted in accordance with the pertinent human germ line variable region sequences in the database; see, e.g., Vbase (http://vbase.mrc-cpe.cam.ac.uk/) hosted by the MRC Centre for Protein Engineering (Cambridge, UK). For example, amino acids considered to potentially deviate from the true germ line sequence could be due to the PCR primer sequences incorporated during the cloning process. Compared to artificially generated human-like antibodies such as single chain antibody fragments (scFvs) from a phage displayed antibody library or xenogeneic mice the human monoclonal antibody of the present disclosure is characterized by (i) being obtained using the human immune response rather than that of animal surrogates, i.e., the antibody has been generated in response to natural Aβ in its relevant conformation in the human body, (ii) having protected the individual or is at least significant for the presence of Aβ, and (iii) since the antibody is of human origin the risks of cross-reactivity against self-antigens is minimized. Thus, in accordance with the disclosure the terms “human monoclonal antibody,” “human monoclonal autoantibody,” “human antibody” and the like are used to denote an Aβ binding molecule which is of human origin, i.e., which has been isolated from a human cell such as a B cell or hybridoma thereof or the cDNA of which has been directly cloned from mRNA of a human cell, for example a human memory B cell. A human antibody is still “human” even if amino acid substitutions are made in the antibody, e.g., to improve binding characteristics.

As used herein, the terms “treat” or “treatment” refer to both therapeutic treatment and prophylactic or preventative measures, wherein the object is to prevent or slow down (lessen) an undesired physiological change, infection, or disorder. Beneficial or desired clinical results include, but are not limited to, alleviation of symptoms, diminishment of extent of disease, stabilized (i.e., not worsening) state of disease, clearance or reduction of an infectious agent in a subject, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the infection, condition, or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, bears, and so on.

### II. Genetic Biomarkers Associated with Rapid Cognitive Decline

Provided herein are methods related to the discovery that certain genetic, markers are associated with a higher probability of rapid decline in cognitive measures and brain glucose metabolism in patients with an early-stage Alzheimer's disease or subjects susceptible to developing Alzheimer's disease.

The present disclosure relates to a method of treating a patient with AD or a subject susceptible to developing AD, comprising assaying a sample obtained from an early-stage AD patient or a subject susceptible to developing AD for the presence of one or more mutations in genes associated with neurodegenerative diseases, e.g., at least one single nucleotide polymorphism (SNP) in, e.g., in the BDNF gene; determining whether the patient or subject is positive for brain amyloid-beta (Aβ), wherein the presence of brain Aβ in combination with one or more mutations in genes associated with neurodegenerative diseases, e.g., at least one single nucleotide polymorphism (SNP) in, e.g., in the BDNF gene, correlates with a prediction of rapid cognitive decline; and treating the patient or subject with early and aggressive therapy appropriate to treat AD with rapid cognitive decline.

The present disclosure relates to a method of treating a patient with AD or a subject susceptible to developing AD, comprising assaying a sample obtained from an early-stage AD patient or a subject susceptible to developing AD for the presence of one or more mutations in genes associated with neurodegenerative diseases, e.g., at least one single nucleotide polymorphism (SNP) in, e.g., in the BDNF gene; determining whether the patient or subject is positive for brain amyloid-beta (Aβ), wherein the presence of brain Aβ in combination with one or more mutations in genes associated with neurodegenerative diseases, e.g., at least one single nucleotide polymorphism (SNP) in, e.g., in the BDNF gene, correlates with a prediction of rapid cognitive decline; and instructing a healthcare provider to administer early and aggressive therapy appropriate to treat AD with rapid cognitive decline.

The present disclosure also relates to a method of treating a patient with AD or a subject susceptible to developing AD, comprising obtaining a sample from an early-stage AD patient or a subject susceptible to developing AD, and submitting the sample for determination of the presence of one or more mutations in genes associated with neurodegenerative diseases, e.g., at least one single nucleotide polymorphism (SNP) in, e.g., in the BDNF gene; ordering a test to determine whether the patient or subject is positive for brain Aβ, wherein the presence of brain Aβ in combination with one or more mutations in genes associated with neurodegenerative diseases, e.g., at least one single nucleotide polymorphism (SNP) in, e.g., in the BDNF gene correlates with a prediction of rapid cognitive decline; and treating the patient or subject with early and aggressive therapy appropriate to treat AD with rapid cognitive decline.

The present disclosure relates to a method of treating a patient with AD or a subject susceptible to developing AD, comprising administering to the patient or subject an anti-Aβ antibody, or antigen-binding fragment thereof, a cholinesterase inhibitor, an N-methyl-D-aspartate receptor antagonist, or any combination thereof, wherein the patient has (a) at least one mutation in a BDNF gene and/or Ptprz1 gene and (b) brain A.

The present disclosure also relates to a method of prognosing a patient with AD or a subject susceptible to developing AD, comprising: (a) assaying a sample obtained from an early-stage AD patient or a subject susceptible to developing AD for the presence of a BDNF gene and/or Ptprz1 gene mutation; and (b) determining whether the patient or subject is positive for brain Aβ; wherein the presence of brain Aβ in combination with the BDNF gene and/or Ptprz1 gene mutation correlates with a prediction of rapid cognitive decline, and indicates a need for rapid, aggressive AD treatment.

The present disclosure relates to a method of predicting the rate of cognitive decline expected in a patient with AD or a subject susceptible to developing AD, comprising assaying a sample obtained from an early-stage AD patient or a subject susceptible to developing AD for the presence of one or more mutations in genes associated with neurodegenerative diseases, e.g., at least one single nucleotide polymorphism (SNP) in, e.g., in the BDNF gene; and determining whether the patient or subject is positive for brain Aβ; wherein the presence of brain Aβ in combination with one or more mutations in genes associated with neurodegenerative diseases, e.g., at least one single nucleotide polymorphism (SNP) in, e.g., in the BDNF gene, correlates with a prediction of rapid cognitive decline, and indicates a need for rapid, aggressive AD treatment.

The present disclosure relates to a method of predicting the rate of cognitive decline expected in a patient with AD or a subject susceptible to developing AD, comprising obtaining a sample from an early-stage AD patient or a subject susceptible to developing AD, and submitting the sample for determination of the presence of one or more mutations in genes associated with neurodegenerative diseases, e.g., at least one single nucleotide polymorphism (SNP) in, e.g., in the BDNF gene; and ordering a test to determine whether the patient or subject is positive for brain Aβ; wherein the presence of brain Aβ in combination with one or more mutations in genes associated with neurodegenerative diseases, e.g., at least one single nucleotide polymorphism (SNP) in, e.g., in the BDNF gene correlates with a prediction of rapid cognitive decline, and indicates a need for rapid, aggressive AD treatment.

In certain aspects, the genetic markers include one or more mutations in genes associated with neurodegenerative diseases, e.g., at least one single nucleotide polymorphism (SNP) in, e.g., in the BDNF gene, the protein tyrosine phosphatase receptor-type, Z polypeptide 1 gene (Ptprz1), or any combination thereof.

The term “allele” refers to alternative forms of a gene or portions thereof. Alleles occupy the same locus or position on homologous chromosomes. When a subject has two identical alleles of a gene, the subject is said to be homozygous for the allele. When a subject has two different alleles of a gene, the subject is said to be heterozygous for the allele. Alleles of a specific gene, e.g., a gene associated with neurodegenerative diseases, e.g., BDNF gene can differ from each other in a single nucleotide. An allele of a gene can also be a form of a gene containing one or more mutations or DNA sequence variants.

A “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term “nucleic acid,” and in particular “DNA molecule” or “RNA molecule,” refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). However, unless specifically stated otherwise, a designation of a nucleic acid includes both the non-transcribed strand referred to above, and its corresponding complementary strand. For purposes of clarity, when referring herein to a nucleotide of a nucleic acid, which can be DNA or an RNA, the terms “adenine”, “cytidine”, “guanine”, and “thymidine” and/or “A”, “C”, “G”, and “T”, respectively, are used. It is understood that if the nucleic acid is RNA, a nucleotide having a uracil base is uridine.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of one or more nucleotides or an insertion of one or more nucleotides relative to a reference allele. Typically, the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNP's can occur in protein-coding nucleic acid sequences, in which case they can give rise to a defective or otherwise variant protein, or genetic disease. Such a SNP can alter the coding sequence of the gene and therefore specify another amino acid (a “missense” SNP) or a SNP can introduce a stop codon either directly (a “nonsense” SNP) or indirectly (by creating or abolishing a splice site). When a SNP does not alter the amino acid sequence of a protein, the SNP is usually “silent.” SNP's can also occur in noncoding regions of the nucleotide sequence. This can result in defective protein expression, e.g., as a result of alternative spicing, or changes in quantitative (spatial or temporal) expression patterns or it may have no effect.

In some embodiments, the BDNF gene mutation comprises at least one copy of Val66Met (A/G) at rs6265. In certain embodiments, the BDNF gene mutation comprises two copies of Val66Met (A/G) at rs6265. In some embodiments, the BDNF gene mutation comprises rs11030104, rs12273363, and/or rs908867 SNP.

In certain embodiments, the Ptprz1 gene mutation comprises at least one copy of “T” allele at rs6946211.

The term “polymorphism” or “polymorphic” refers to the coexistence of more than one form of a gene or portion thereof. A portion of a gene in which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurring most frequently in a selected population is often referred to as the reference and/or wild-type form. Other allelic forms are typically designated or alternative or variant alleles. Diploid organisms can be homozygous or heterozygous for allelic forms. A diallelic or biallelic polymorphism has two forms. A “polymorphic gene” refers to a gene having at least one polymorphic region.

The term “polymorphic nucleotide” or “polymorphic marker” refers to one or more nucleotides that can be used in predicting faster decline in cognitive measures and brain glucose metabolism in a patient with an early-stage AD or a subject susceptible to developing AD. The polymorphic marker can be a SNP.

The term “primer” (or “probe”) refers to a length of single-stranded nucleic acids, which is used in combination with a polymerase to amplify or extend a region from a template nucleic acid. Primers are generally short (e.g., 15-30 bases), but can be longer if required. The primer must contain a sequence which hybridizes with the template nucleic acid under the conditions used. Primers can be used singly, that is, a single primer consisting only of a single sequence can be used in the amplification reaction, and will produce one copy of one strand of the template per cycle of amplification. This can be done in situations where a large number of copies is not required, or where only one strand is to be copied (e.g., in producing antisense products), or if the sequence at the other end of the template is unsuitable for choosing a second primer. More generally, a pair of primers is used in an amplification reaction. The two are of different sequences, and are used in combination, and produce a copy of each template strand per cycle of amplification. The two different primers should not be complementary to each other, or they will hybridize to each other rather than the template, and the polymerase will then be unable to make a copy of the template. Commonly, the two primers are chosen from sequence at the 5′ end of each of the two complementary strands of the template nucleic acid. “Primer” also refers to a short nucleotide sequence complementary to the sequence of nucleotides 5′ or 3′ to the polymorphic nucleotide targeted for detection by an extension reaction. The “primer” is designed such that the polymorphic marker is detected by the methods disclosed herein.

The primer can be sequence specific which means a primer which specifically hybridizes with a nucleic acid sequence present in one or more alleles of a genetic locus or their complementary strands but not a nucleic acid sequence present in all the alleles of the locus. The sequence-specific primer does not hybridize with alleles of the genetic locus that do not contain the sequence polymorphism under the conditions used in the amplification method. The primer of the disclosure comprises a sequence that flanks and/or preferably overlaps, at least one polymorphic site occupied by any of the possible variant nucleotides. The nucleotide sequence of an overlapping probe can correspond to the coding sequence of the allele or to the complement of the coding sequence of the allele.

The term “hybridization probe” or “probe” as used herein is intended to include oligonucleotides which hybridize in a base-specific manner to a complementary strand of a target nucleic acid. Such probes include peptide nucleic acids, and described in Nielsen et al., Science 254: 1497-1 500 (1991). Probes can be any length suitable for specific hybridization to the target nucleic acid sequence. The most appropriate length of the probe can vary depending on the hybridization method in which it is being used; for example, particular lengths may be more appropriate for use in microfabricated arrays, while other lengths may be more suitable for use in classical hybridization methods. Such optimizations are known to the skilled artisan. Suitable probes can range form about 5 nucleotides to about 30 nucleotides in length. For example, probes can be 5, 6, 8, 10, 12, 14, 16, 18, 20, 22, 24, 25, 26, 28 or 30 nucleotides in length. The probe of the disclosure comprises a sequence that flanks and/or preferably overlaps, at least one polymorphic site occupied by any of the possible variant nucleotides. The nucleotide sequence of an overlapping probe can correspond to the coding sequence of the allele or to the complement of the coding sequence of the allele.

As used herein, the term “specifically hybridizes” or “specifically detects” or “specific hybridization” refers to the ability of a nucleic acid molecule of the disclosure to stably hybridize to either strand of, for example, the BDNF gene polymorphic region containing one allele but not to or less stably than a different allele under the same hybridization conditions. This selectivity is based on the nucleotide sequence of the probe, which is complementary to the target nucleic acid sequence or sequences.

A “haplotype” is a term denoting the collective allelic state of a number of closely linked polymorphic loci (i.e., SNPs) on a chromosome. This non-random association of alleles renders these markers tightly linked. Tight linkage (linkage disequilibrium, LD) can induce strong correlation between the genetic histories of neighboring polymorphisms and, when LD is very high, alleles of linked markers can sometimes be used as surrogates for the state of nearby loci. “Determining the subject's haplotype” refers to determining a subject's genetic profile or the unique chromosomal distribution of polymorphic nucleotides or polymorphic markers in or in the vicinity of, for example, the BDNF gene.

As used herein the term, “linkage disequilibrium” refers to co-inheritance of two or more alleles at frequencies greater than would be expected from the separate frequencies of occurrence of each allele in the corresponding control population. The expected frequency of occurrence of two or more alleles that are inherited independently is the population frequency of the first allele multiplied by the population frequency of the second allele. Alleles or polymorphisms that co-occur at expected frequencies are said to be in linkage equilibrium.

One of skill in the art would be able to determine additional polymorphic alleles in linkage disequilibrium with the polymorphic markers of the invention. There are numerous statistical methods to detect linkage disequilibrium, including those found in Terwilliger, Am J Hum Genet, 56:777-787 (1995); Devlin, N. et al., Genomics, 36:1-16, (1996); Lazzeroni, Am J Hum Genet, 62:159-170, (1998); Service, et al. Am J HUM Genet, 64:1728-1738 (1999); McPeek and Strahs, and Am J Hum Genet, 65:858-875 (1999), all of which are herein incorporated by reference in their entirety.

The disclosure further provides allele-specific oligonucleotides that hybridize to a gene comprising a single nucleotide polymorphism or to the complement of the gene. Such oligonucleotides will hybridize to one allele of the nucleic acid molecules described herein but not a different allele. The oligonucleotides of the invention also include probes and primers which hybridize to regions 5′ and 3′ of the polymorphism.

### III. Therapy for Treatment of Alzheimer's Disease

The present disclosure provides the method as described herein, wherein the therapy includes but it is not limited to administration of an anti-Aβ antibody, or antigen-binding fragment thereof, a cholinesterase inhibitor, an N-methyl-D-aspartate receptor antagonist, or any combination thereof.

As disclosed herein, an anti-Aβ antibody or antigen-binding fragment thereof that binds to the same epitope as BIIB037 antibody, wherein BIIB037 antibody binds to an epitope comprising amino acids 3-6 of Aβ. BIIB037 antibody is described as NI-101.12F6A described in the International Publication No. WO2008/081008 incorporated herein by reference in its entirety.

Anti-Aβ antibodies or antigen-binding fragments thereof, as described herein, specifically bind to Aβ and epitopes thereof and to various conformations of Aβ and epitopes thereof. For example, disclosed herein are antibodies or antigen-binding fragments thereof that selectively bind to Aβ aggregates. As used herein, reference to an antibody that “selectively binds,” “specifically binds,” or “preferentially binds” Aβ refers to an antibody that does not bind other unrelated proteins. An antibody that “selectively binds” or “specifically binds” Aβ conformer refers to an antibody that does not bind all conformations of Aβ, i.e., does not bind at least one other Aβ conformer. For example, disclosed herein are antibodies or antigen-binding fragments thereof that can distinguish among monomeric and aggregated forms of Aβ, i.e., bind to Aβ fibril but not Aβ monomer.

In certain embodiments, an anti-Aβ antibody or antigen-binding fragment, variant, or derivative thereof useful in the methods provided herein has an amino acid sequence that has at least about 80%, about 85%, about 88%, about 89%, about 90%, about 91%, about 92%, about 93%, about 94%, or about 95% sequence identity to the amino acid sequence of BIIB037 antibody. In a further embodiment, the binding molecule shares at least about 96%, about 97%, about 98%, about 99%, or 100% sequence identity to BIIB037 antibody. In certain embodiments, an anti-Aβ antibody or antigen-binding fragment, variant, or derivative thereof specifically binds to the same Aβ epitope as BIIB037 antibody. In some embodiments, an anti-Aβ antibody or antigen-binding fragment, variant, or derivative thereof comprises an immunoglobulin heavy chain variable region (VH) and an immunoglobulin light chain variable region (VL), wherein the VH comprises amino acid sequence at least 80%, 85%, 90% 95% or 100% identical to SEQ ID NO: 1 and the VL comprises amino acid sequence at least 80%, 85%, 90% 95% or 100% identical to SEQ ID NO: 2, as shown in Table 2.

In some embodiments, an anti-Aβ antibody or antigen-binding fragment, variant, or derivative thereof comprises VH and a VL, wherein the VH comprises amino acid sequence identical to, or identical except for one, two, three, four, five, or more amino acid substitutions to SEQ ID NO: 1, and the VL comprises amino acid sequence identical to, or identical except for one, two, three, four, five, or more amino acid substitutions to SEQ ID NO: 2, as shown in Table 2.

Some embodiments include an anti-Aβ antibody or antigen-binding fragment, variant, or derivative thereof useful in the methods provided herein which comprises a VH, where one or more of the VHCDR1, VHCDR2 or VHCDR3 regions of the VH are at least 80%, 85%, 90%, 95% or 100% identical to one or more reference heavy chain VHCDR1, VHCDR2 and/or VHCDR3 amino acid sequences of one or more of: SEQ ID NO: 3, SEQ ID NO: 4, SEQ ID NO: 5, as shown in Table 3.

Further disclosed is an anti-Aβ antibody or antigen-binding fragment, variant, or derivative thereof useful in the methods provided herein which comprises a VH, where one or more of the VHCDR1, VHCDR2 or VHCDR3 regions of the VH are identical to, or identical except for four, three, two, or one amino acid substitutions, to one or more reference heavy chain VHCDR1, VHCDR2 or VHCDR3 amino acid sequences of one or more of: SEQ ID NO: 3, SEQ ID NO: 4, SEQ ID NO: 5, as shown in Table 3.

Also disclosed is an anti-Aβ antibody or antigen-binding fragment, variant, or derivative thereof useful in the methods provided herein which comprises a VL, where one or more of the VLCDR1, VLCDR2 or VLCDR3 regions of the VL are at least 80%, 85%, 90%, 95% or 100% identical to one or more reference heavy chain VLCDR1, VLCDR2 or VLCDR3 amino acid sequences of one or more of: SEQ ID NO: 6, SEQ ID NO: 7, SEQ ID NO: 8, as shown in Table 3.

In some embodiments, an anti-Aβ antibody or antigen-binding fragment, variant, or derivative thereof useful in the methods provided herein comprises a VL, where one or more of the VLCDR1, VLCDR2 or VLCDR3 regions of the VL are identical to, or identical except for four, three, two, or one amino acid substitutions, to one or more reference heavy chain VLCDR1, VLCDR2 or VLCDR3 amino acid sequences of one or more of: SEQ ID NO: 6, SEQ ID NO: 7, SEQ ID NO: 8, as shown in Table 3.

In some embodiments, an anti-Aβ antibody or antigen-binding fragment, variant, or derivative thereof useful in the methods provided herein comprises BIIB037 antibody.

TABLE 2
BIIB037 antibody VH and VL
amino acid sequences

| VH | VL |
|---|---|
| QVQLVESGGGVV | DIQMTQSPSSLS |
| QPGRSLRLSCAA | ASVGDRVTITCR |
| SGFAFSSYGMHW | ASQSISSYLNWY |
| VRQAPGKGLEWV | QQKPGKAPKLLI |
| AVIWFDGTKKYY | YAASSLQSGVPS |
| TDSVKGRFTISR | RFSGSGSGTDFT |
| DNSKNTLYLQMN | LTISSLQPEDFA |
| TLRAEDTAVYYC | TYYCQQSYSTPL |
| ARDRGIGARRGP | TFGGGTKVEIKR |
| YYMDVWGKGTTV | SEQ ID NO: 2 |
| TVSS | |
| SEQ ID NO: 1 | |

TABLE 3
BIIB037 Antibody VH and VL CDR1, CDR2,
and CDR3 amino acid sequences

| VHCDR1 | VHCDR2 | VHCDR3 | VLCDR1 | VLCDR2 | VLCDR3 |
|---|---|---|---|---|---|
| SYGMH | VIWFDG | DRGIGA | RASQSI | AASSLQ | QQSYST |
| SEQ ID | TKKYYT | RRGPYY | SSYLN | S | PLT |
| NO: 3 | DSVKG | MDV | SEQ ID | SEQ ID | SEQ ID |
| | SEQ ID | SEQ ID | NO: 6 | NO: 7 | NO: 8 |
| | NO: 4 | NO: 5 | | | |

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

When discussed herein whether any particular polypeptide, including the constant regions, CDRs, VH domain or VL domains disclosed herein, is at least about 65%, about 70%, about 75%, about 80%, about 85%, about 90%, about 91%, about 92%, about 93%, about 94%, about 95%, about 96%, about 97%, about 98%, about 99%, or even about 100% identical to another polypeptide, the comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

Also included for use in the methods described herein are polypeptides encoding anti-Aβ antibodies, or antigen-binding fragments, variants, or derivatives thereof as described herein, polynucleotides encoding such polypeptides, vectors comprising such polynucleotides, and host cells comprising such vectors or polynucleotides, all for producing anti-Aβ antibodies, or antigen-binding fragments, variants, or derivatives thereof for use in the methods described herein.

Suitable biologically active variants of anti-Aβ antibodies as described herein can be used in the methods of the disclosure. Such variants will retain the desired binding properties of the parent anti-Aβ antibody. Methods for making antibody variants are generally available in the art.

Cholinesterase inhibitors, as described herein work by inhibiting the breakdown of acetylcholine, an important neurotransmitter associated with memory, by blocking the enzyme acetylcholinesterase. For example, donepezil, galantamine, rivastigmine and tacrine are cholinesterase inhibitors. (See, e.g., Birks J., Cochrane Database of Systematic Reviews 2006, Issue 1. Art. No.: CD005593).

An N-methyl-D-aspartate (NMDA) receptor antagonists, as described herein, e.g., memantine, work by regulating the activity of glutamate, a chemical messenger involved in learning and memory. Memantine protects brain cells against excess glutamate, released in large amounts by cells damaged by Alzheimer's disease and other neurological disorders. Attachment of glutamate to cell surface “docking sites” called NMDA receptors permits calcium to flow freely into the cell. Over time, this leads to chronic overexposure to calcium, which can speed up cell damage. Memantine prevents this destructive chain of events by partially blocking the NMDA receptors (See, e.g., Danysz et al., Neurotoxicity Research (2):85-97 (2000)).

### IV. Methods of Treatment of Alzheimer's Disease and Predicting Rate of Cognitive Decline

The methods of the disclosure can be characterized as comprising detecting, in a sample obtained from an early-stage AD patient or a subject susceptible to developing AD, the presence or absence of a specific allelic variant of one or more polymorphic regions of a gene or genes associated with neurodegenerative diseases, including but not limited to BDNF and/or protein tyrosine phosphatase, receptor-type, Z polypeptide 1 (Ptprz1).

The sample from an early-stage AD patient or a subject susceptible to developing AD comprises fresh, frozen, or preserved tissue, a biopsy, an aspirate, blood or any blood constituent, a bodily fluid, cells, or any combination thereof.

The sample can be any appropriate sample including but not limited to the target SNPs (or target polypeptides). The sample can be and obtained from any cell type, tissue or bodily fluid (e.g., blood, serum, plasma, urine, saliva, tears, vaginal secretion, lymph fluid, cerebrospinal fluid, mucosa secretion, peritoneal fluid, ascitic fluid, or body exudates) of a subject.

The samples can, for example, be obtained from a subject's bodily fluid (e.g., blood or ay blood constituent) by known techniques (e.g., venipuncture). Alternatively, the sample to be analyzed by any of the methods described can be dry samples (e.g., hair or skin). The sample analysis can also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (See, e.g., Nuovo, G. J., PCR in situ hybridization: protocols and applications, Raven Press, N.Y. (1992)).

The sample can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to yet another healthcare provider, healthcare benefits provider or the patient. Similarly, assaying a sample obtained from an early-stage AD patient or a subject susceptible to developing AD for the presence of a neurodegenerative disease specific gene mutation, e.g., the BDNF gene mutation, and evaluation of the results can be performed by one or more healthcare providers, healthcare benefits providers, and/or clinical laboratories.

As used herein, the term “healthcare provider” refers to individuals or institutions which directly interact and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being. In certain aspects a clinical laboratory can be “centralized” or “local”, meaning that a small number or a single laboratory makes all measurements of samples submitted from all outside sources. In other aspects, multiple clinical laboratories, also referred to as “satellite” or “global” laboratories, can be validated to all provide standard, reliable results that can be easily compared.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer early and aggressive therapy appropriate to treat AD with rapid cognitive decline. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy or therapeutic agent (e.g., an anti-Aβ antibody, or antigen-binding fragment thereof, a cholinesterase inhibitor, an N-methyl-D-aspartate receptor antagonist, or any combination thereof), commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition a healthcare benefits providers can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples.

The above enumerated actions can be performed by a healthcare provider, healthcare benefits provider, or patient automatically using a computer-implemented method (e.g., via a web service or stand-alone computer system).

As used herein the term “directing a healthcare provider” includes orally directing a healthcare provider, or directing a healthcare provider by using a written order, or both.

The sample, as described herein, can be sequenced to identify homozygous or heterozygous loci of interest, which are the loci of interest analyzed on the template DNA obtained from the sample.

The locus of interest to be copied can be within a coding sequence or outside of a coding sequence. One or more loci of interest that are to be copied are within a gene. In certain embodiments, the template DNA that is copied is a locus or loci of interest that is within a genomic coding sequence, either intron or exon. In some embodiments, exon DNA sequences are copied. The loci of interest can be sites where mutations are known to cause disease or predispose to a disease state. In some embodiments, the loci of interest can be sites of SNPs. Alternatively, the loci of interest that are to be copied can be outside of the coding sequence, for example, in a transcriptional regulatory region, and especially a promoter, enhancer, or repressor sequence.

Any method that provides information on the sequence of a nucleic acid can be used to determine the sequence of locus of interest, including but not limited to allele specific PCR, PCR, gel electrophoresis, ELISA, mass spectrometry, MALDI-TOF mass spectrometry hybridization, primer extension, fluorescence detection, fluorescence resonance energy transfer (FRET), fluorescence polarization, DNA sequencing, Sanger dideoxy sequencing, DNA sequencing gels, capillary electrophoresis on an automated DNA sequencing machine, microchannel electrophoresis, microarray, southern blot, slot blot, dot blot, single primer linear nucleic acid amplification, as described in U.S. Pat. No. 6,251,639, SNP-IT, GeneChips,® HuSNP, BeadArray, TaqMan® assay, flap endonuclease assay (e.g., Invader® assay), MassExtend®, or MassCleave™ (hMC) method.

In practicing the present disclosure, the subject's sample can be assayed for the presence of one or more mutations in genes associated with neurodegenerative diseases, e.g., at least one single nucleotide polymorphism (SNP) in, e.g., in the BDNF gene.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension,” or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridisation. These methods are largely reliant on the discriminatory techniques of Affymetrix (Santa Clara, Calif.) and Nanogen Inc. (San Diego, Calif.) are binding of oligonucleotides to target sequences containing the SNP of interest. The particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridisation require target amplification by methods such as PCR to increase sensitivity and specificity (See, for example U.S. Pat. No. 5,679,524, PCT publication WO 98/59066, PCT publication WO 95/12607). US Application 20050059030 (incorporated herein in its entirety) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Application 20050042608 (incorporated herein in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of 40,000 SNPs per day, using mass code tags.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′ end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analysing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 86:2766-2770 (1989)) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bi-directional dideoxy fingerprinting (in which the dideoxy termination reaction is performed simultaneously with two opposing primers), and Fluorescent PCR-SSCP (in which PCR products are internally labelled with multiple fluorescent dyes, can be digested with restriction enzymes, followed by SSCP, and analysed on an automated DNA sequencer able to detect the fluorescent dyes).

Other methods which utilise the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electrophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonymous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the disclosure.

Non-invasive detection and quantitation of amyloid deposits in the brain has been used to develop anti-amyloid therapies. Direct imaging of amyloid load in vivo in patients with AD is useful for the early diagnosis of AD and the development and assessment of treatment strategies. The small molecule approach for amyloid imaging has so far been the most successful. Some of the promising compounds used to image amyloid are based on Congo red, thioflavin, and stilbene, and compounds such as [18F]1-(6-((2-fluoroethyl)-methyl)amino)naphthalen-2-yl)ethylidene)malononitrile ([¹⁸F]FDDNP). Amyloid-β (Aβ) imaging with N-methyl-^{u}C-(4′-methylamino-phenyl)-6-hydroxy-benzothiazole (^{u}C-6-OH-BTA-1; also known as ^{U}C-PIB) has also been used. The binding of different derivatives of Congo red and thioflavin has been studied in human autopsy brain tissue and in transgenic mice. Two compounds in advanced testing are fluorine-18-labelled Amyvid™ (florbetapir) from Eli Lilly ((E)-4-(2-(6-(2-(2-(2-[¹⁸F]fluoroethoxy)ethoxy)ethoxy)pyridin-3-yl)vinyl)-N-methylaniline, and flutemetamol from GE (2-(3-fluoro-4-(methylamino)phenyl)benzo[d]thiazol-6-ol). See e.g., International Publication No. WO 2013/040183 and U.S. Pat. No. 7,687,052 B2.

Researchers have also been using [¹⁸F]-fluorodeoxyglucose ([¹⁸F]-FDG) positron emission tomography (PET) and magnetic resonance imaging (MRI) to detect and track changes in brain function and structure which precede the onset of brain disorder symptoms in cognitively normal persons who are at risk for developing brain disorders such as Alzheimer's disease. See e.g., International Publication No. WO 2006/009887 A2

The uptake pattern and the amount of Aβ present in the brain can also be visualized with PET using the PET radioligand N-methyl-[¹¹C]2-(4-methylaminophenyl)-6-hydroxybenzothiazole (also known as [¹¹C]6-OH-BTA-1 and [¹¹C]PiB). [¹¹C]PiB binds to amyloid beta (AP) which accumulates pathologically in Alzheimer's Disease (AD). N-methyl-³H}2-[4′-(methylamino)phenyl]6-hydroxybenzothiazole ([³H]PIB) is also suitable for use as an amyloid imaging agent for use with the methods described herein. See e.g., U.S. Publication No. 2011/0160543 A1.

### V. Compositions and Administration Methods

The methods of preparing and administering anti-Aβ antibodies, or antigen-binding fragments, variants, or derivatives thereof to a subject in need thereof are well known to or are readily determined by those skilled in the art. The route of administration of an anti-Aβ antibody, or antigen-binding fragment, variant, or derivative thereof, a cholinesterase inhibitor, an N-methyl-D-aspartate receptor antagonist, or any combination thereof, can be, for example, peripheral, oral, parenteral, by inhalation or topical.

As discussed herein, anti-Aβ antibodies, or antigen-binding fragments, variants, or derivatives thereof, a cholinesterase inhibitor, an N-methyl-D-aspartate receptor antagonist can be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an anti-Aβ antibody, or antigen-binding fragment, variant, or derivative thereof, a cholinesterase inhibitor, an N-methyl-D-aspartate receptor antagonist, or any combination shall be held to mean an amount sufficient to achieve effective binding to a target and to achieve a benefit, e.g., treating the patient with AD or subject susceptible to developingAD with early and aggressive therapy appropriate to treat AD with rapid cognitive decline.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Prevention of the action of microorganisms can be achieved by various antibacterial and antifungal agents, for example, parabens, chlorobutanol, phenol, ascorbic acid, thimerosal and the like. In many cases, isotonic agents can be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

Parenteral formulations can be a single bolus dose, an infusion or a loading bolus dose followed with a maintenance dose. These compositions can be administered at specific fixed or variable intervals, e.g., once a day, or on an “as needed” basis.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. Furthermore, the pharmaceutical composition of the disclosure can comprise further agents such as dopamine or psychopharmacologic drugs, depending on the intended use of the pharmaceutical composition. Furthermore, the pharmaceutical composition can also be formulated as a vaccine, for example, if the pharmaceutical composition of the disclosure comprises an anti-Aβ antibody for passive immunization.

Certain pharmaceutical compositions, as disclosed herein, can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an anti-Aβ antibody, or fragment, variant, or derivative thereof, a cholinesterase inhibitor, or an N-methyl-D-aspartate receptor antagonist, to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

The term “peripheral administration” as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g., acetate, phosphate or citrate buffer), a surfactant (e.g., polysorbate), optionally a stabilizer agent (e.g., human albumin), etc. Preparations for peripheral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In the subject disclosure, pharmaceutically acceptable carriers include, but are not limited to, 0.01-0.1 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

The practice of the disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Roitt, I., Brostoff, J. and Male D., Immunology, 6^{th} ed. London: Mosby (2001); Abbas A., Abul, A. and Lichtman, A., Cellular and Molecular Immunology, Ed. 5, Elsevier Health Sciences Division (2005); and Harlow and Lane, Antibodies: A Laboratory Manual, Cold Spring Harbor Press (1988).

Having now described the disclosure in detail, the same will be more clearly understood by reference to the following examples, which are included herewith for purposes of illustration only and are not intended to be limiting of the disclosure. All patents and publications referred to herein are expressly incorporated by reference in their entireties.

### EXAMPLES

Detailed descriptions of conventional methods, such as those employed herein can be found in the cited literature. Unless indicated otherwise below, identification of Aβ-specific B cells and molecular cloning of anti-Aβ antibodies displaying specificity of interest as well as their recombinant expression and functional characterization has been or can be performed as described in the Examples and Supplementary Methods section of international applications PCT/EP2008/000053 published as WO2008/081008, and international applications PCT/EP2009/009186 published as WO2010/069603, the disclosure content of which is incorporated herein by reference in its entirety.

### Example 1

### Genetic and Image Biomarkers Associated with Decline in Multiple Cognitive Measures and Brain Glucose Metabolism in Populations of Early Alzheimer's Disease

Statistical analyses of the Australian Imaging, Biomarker and Lifestyle (AIBL) Flagship Study of Ageing data revealed that a mutation, Val66Met at rs6265, in the BDNF gene is strongly associated with faster cognitive decline with the presence of brain amyloid in the normal-to-early Alzheimer's disease population as described in Lim Y. et al., Neurobiology of Aging, article in press, p. 1-8 (2013).

This example describes the study using the Alzheimer's disease Neuroimaging Initiative (ADNI) data.

Data Acquisition:

The genetic (Plink format) and clinical data were downloaded from the ADNI website. The genetic data were pre-processed following the procedures applied by the ADNI genetic core as described in Shen L, et al., Neuroimage 53: 1051-1063 (2010). The clinical datasets, which contain demographic variables, neuro-battery tests results and clinical and functional longitudinal measures, were merged. Summary statistical analyses were conducted. The results were compared to published results to ensure data quality.

Image Data Analysis:

Pittsburgh compound B (PiB)-positron emission tomography (PET), florbetapir (AV45) and [(18)F]fluorodeoxyglucose (FDG)-PET data were analyzed by Synarc Inc. For PiB-PET and AV45, cut-off values of positive calls are determined following ADNI PET core's suggestions. FDG-PET data were analyzed following procedures introduced by the ADNI PET core and Landou et al., Neurobiol Aging 32: 1207-1218 (2011).

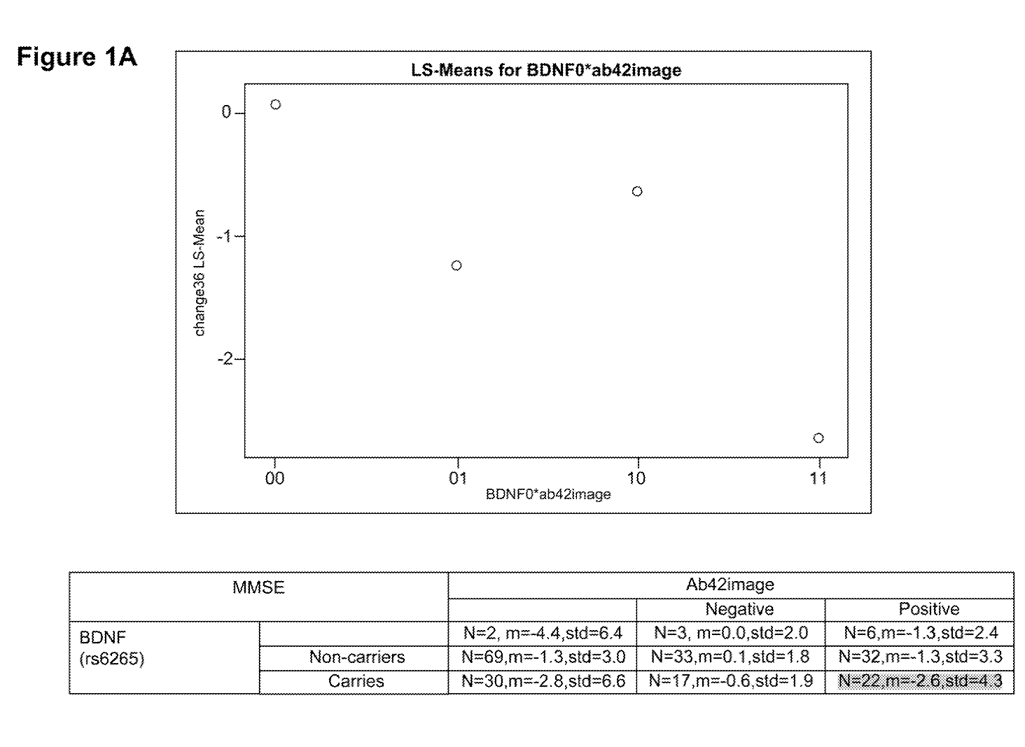

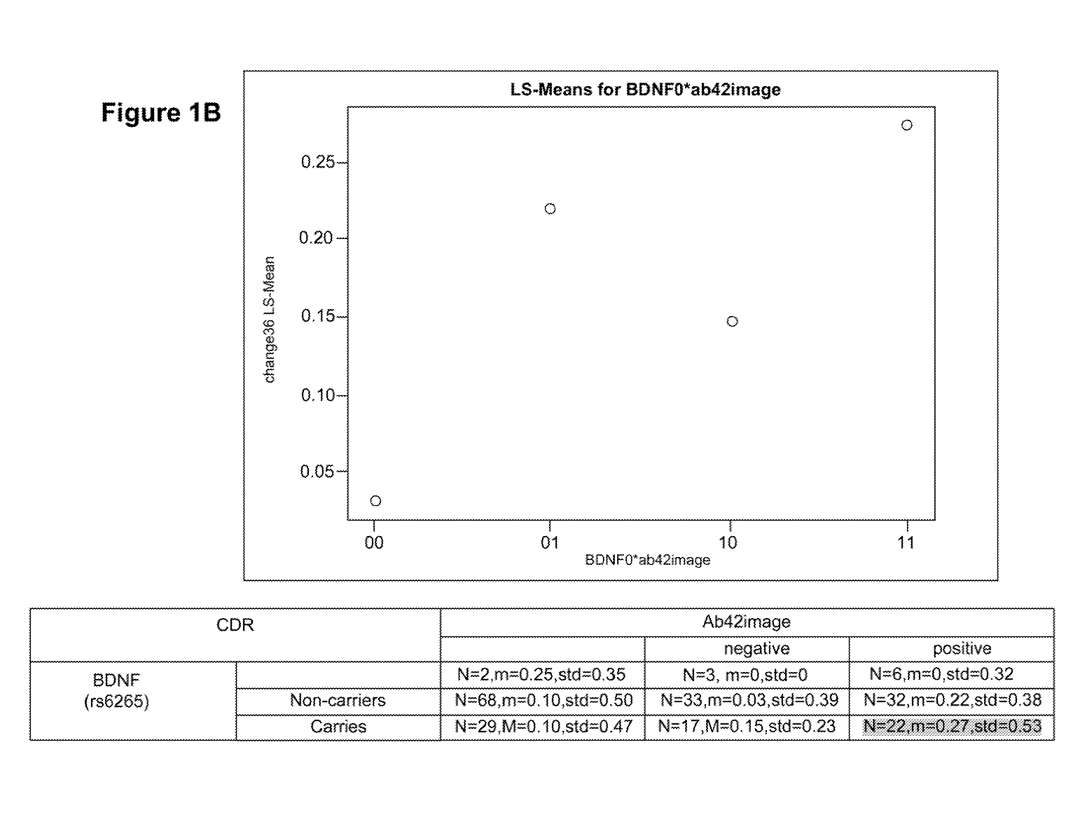

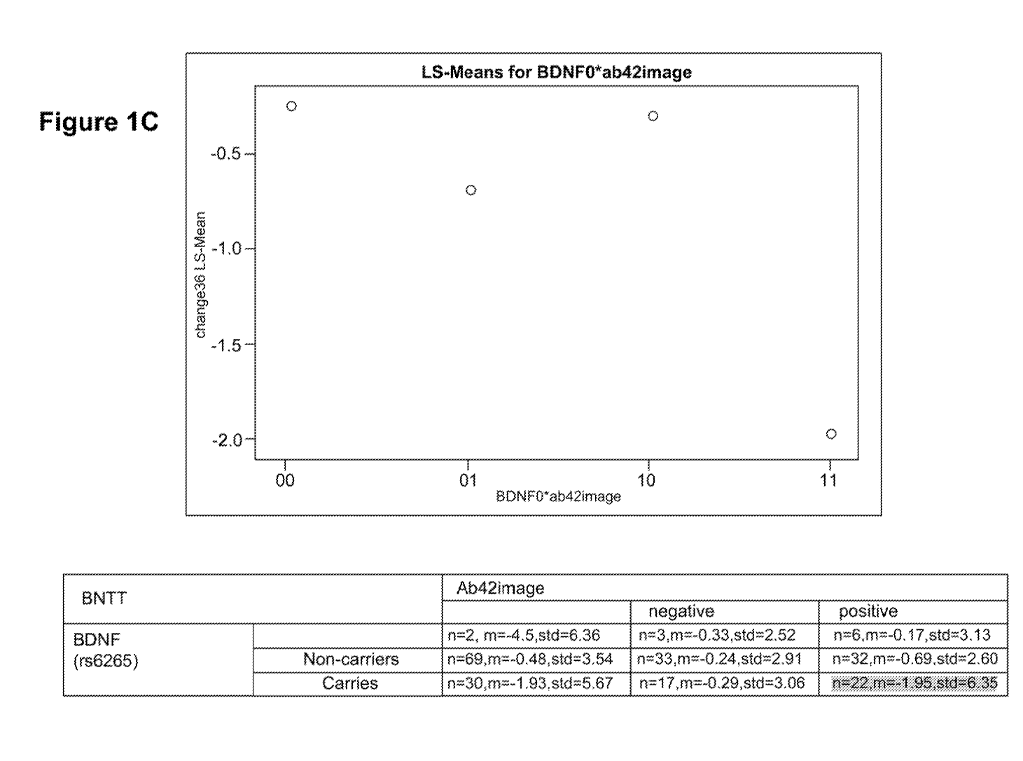

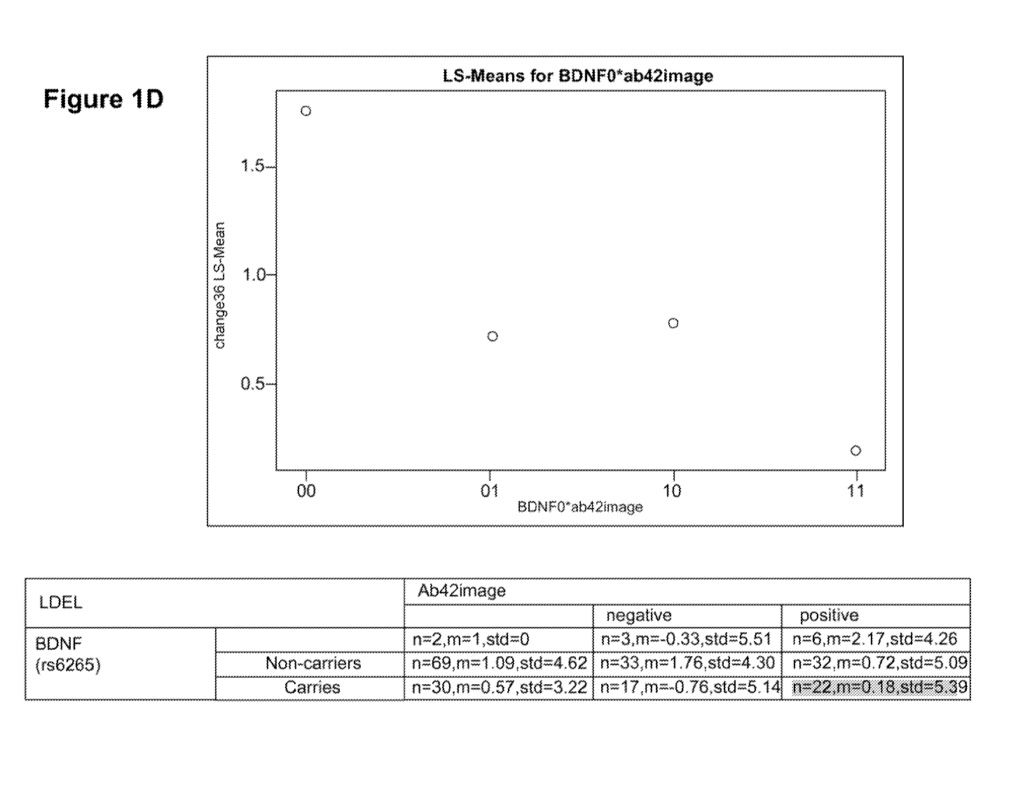

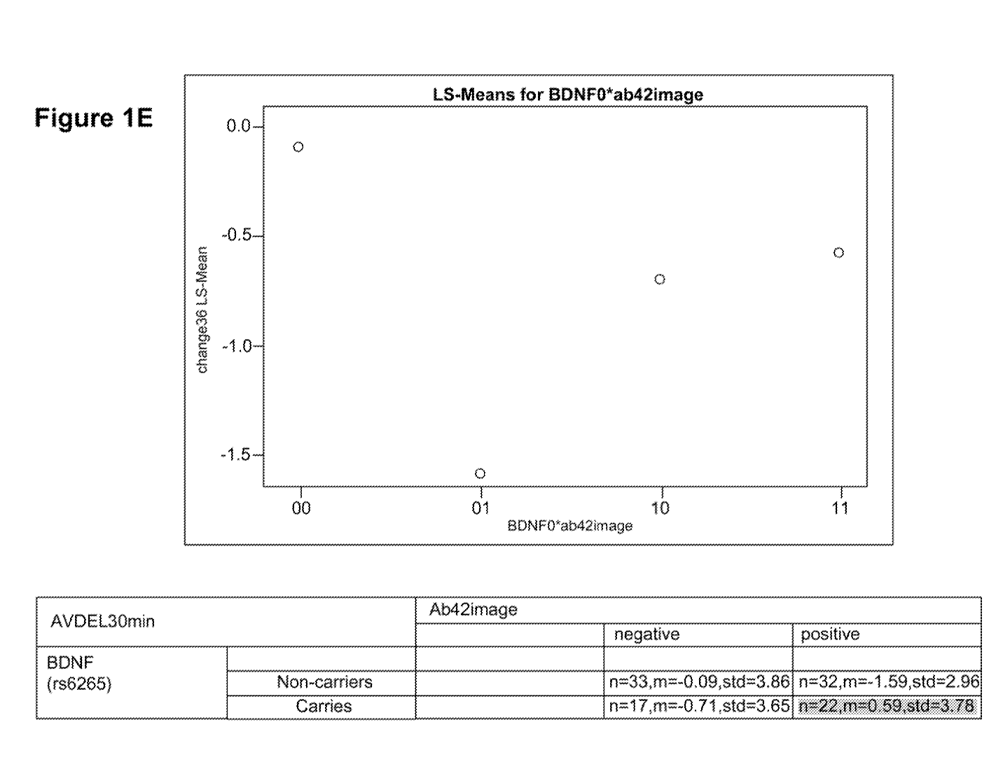

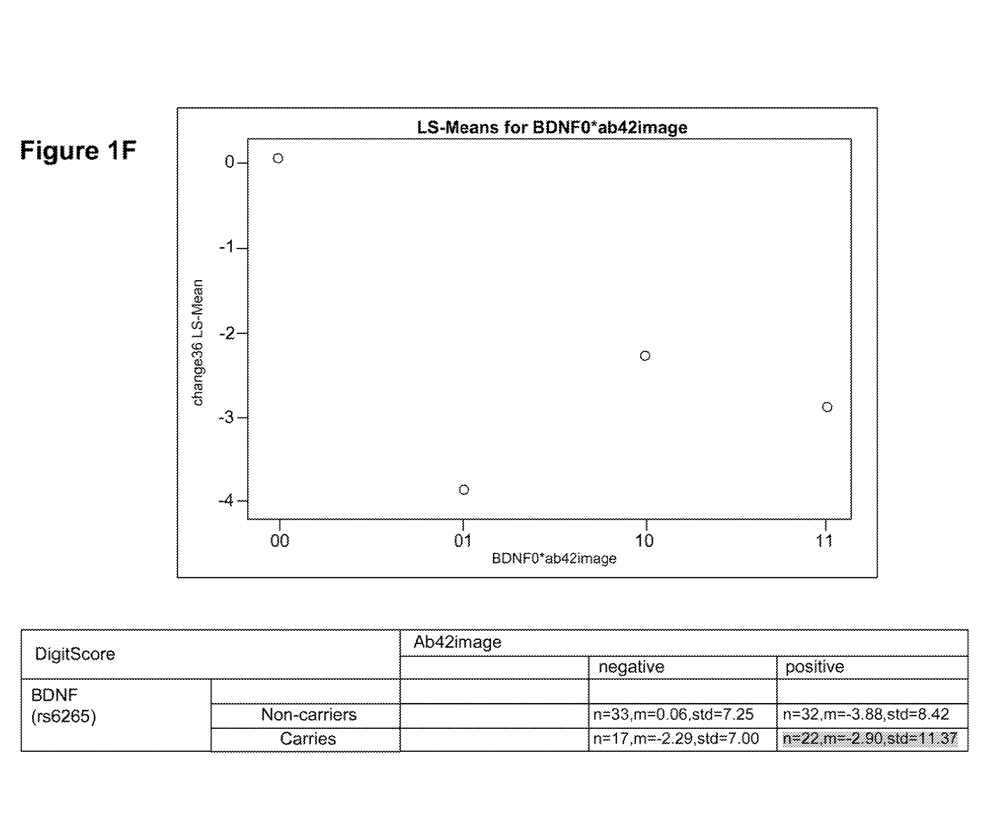

FIGS. 1A to 1E show that a patient positive for both brain Aβ and at least one copy of the Val66Met mutation has a faster 36 month cognitive decline than a patient negative for either brain Aβ or a Val66Met mutation, based on the evaluation of multiple cognitive measures. FIGS. 1A to 1D show the strongest statistical evidence.

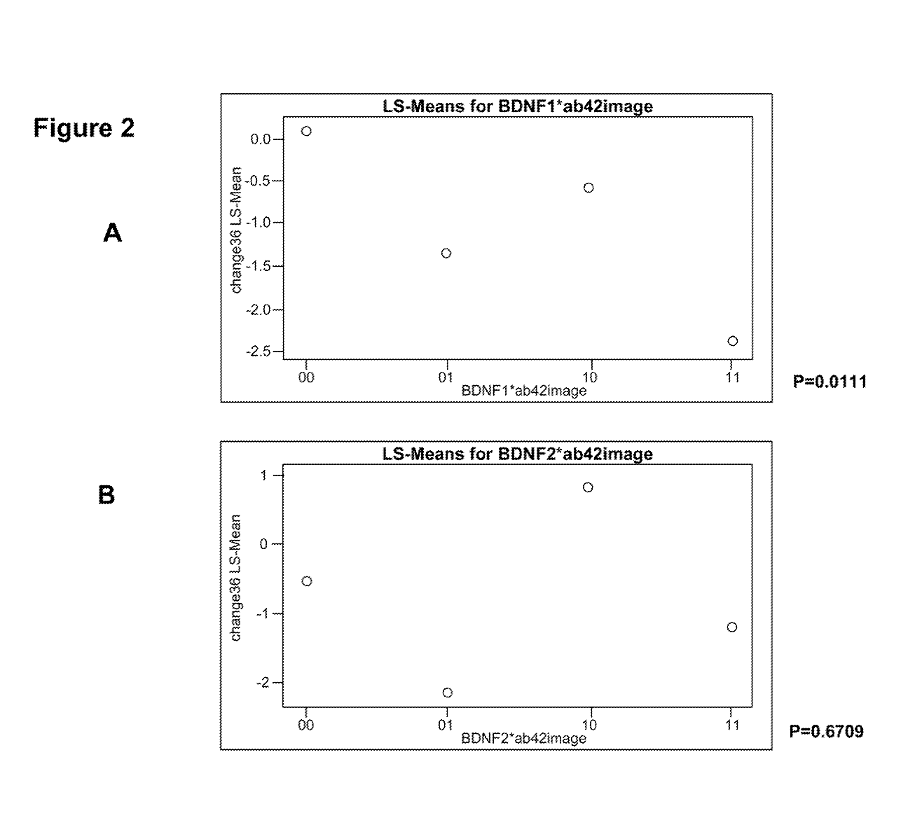

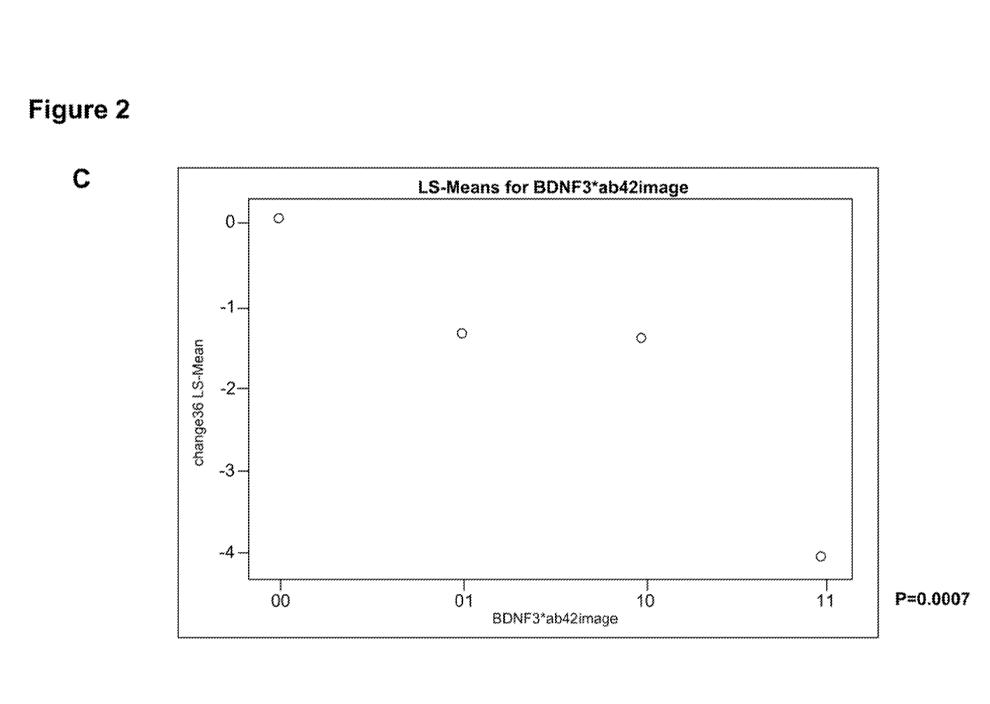

FIGS. 2A to 2C show that a patient positive for both brain Aβ and (A) rs11030104, (B) rs12273363, or (C) rs908867 has a faster 36 month cognitive decline than a patient negative for either brain Aβ or mutation, based on the evaluation of mini-mental state examination.

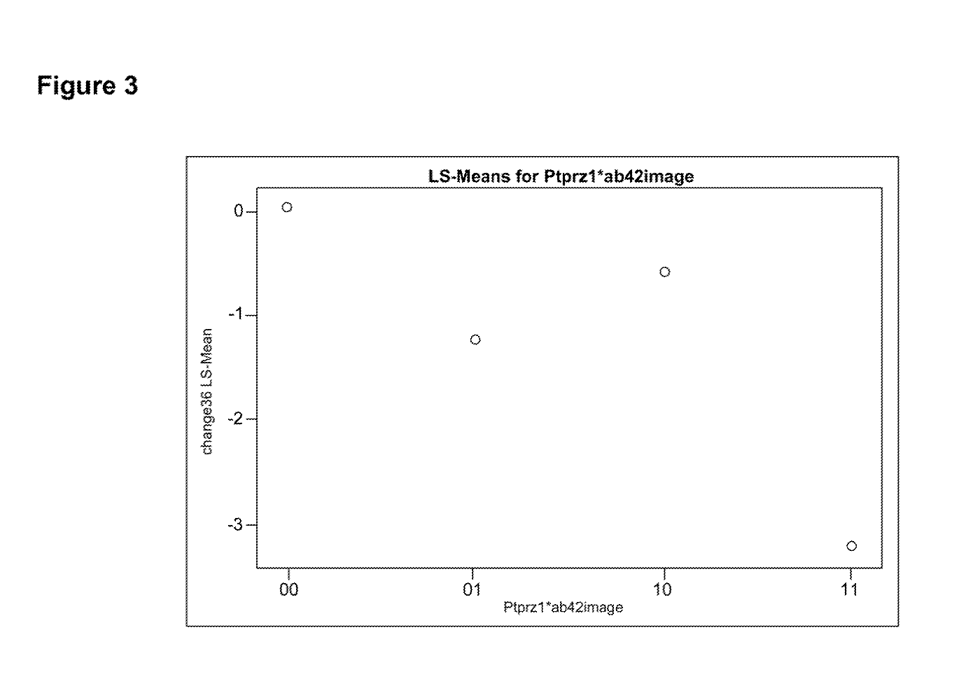

FIG. 3 shows that a patient positive for both brain Aβ and at least one copy of “T” allele at rs6946211 in the Ptprz1 gene has a faster 36 month cognitive decline than a patient negative for either brain Aβ or mutation, based on the evaluation of mini-mental state examination.

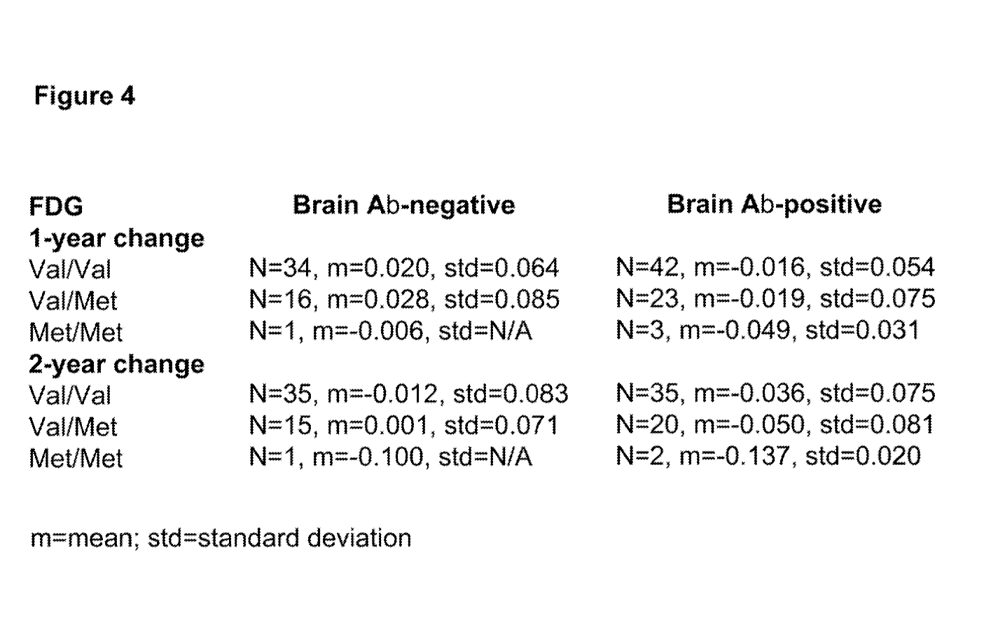

FIG. 4 shows that a patient positive for both brain Aβ and at least one copy of the Val66Met mutation has a faster decline in brain glucose metabolism, as measured by FDG-PET, than a patient negative for either brain Aβ or a Val66Met mutation.

The foregoing description of the specific aspects will so fully reveal the general nature of the disclosure that others can, by applying knowledge within the skill of the art, readily modify and/or adapt for various applications such specific aspects, without undue experimentation, without departing from the general concepts provided. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A method of treating a patient with Alzheimer's disease (AD) or a subject susceptible to developing AD, comprising:
(a) assaying a sample obtained from an early-stage AD patient or a subject susceptible to developing AD for the presence of a brain-derived neurotrophic factor (BDNF) gene mutation;
(b) determining whether the patient or subject is positive for brain amyloid-beta (Aβ), wherein the presence of brain Aβ in combination with the BDNF gene mutation correlates with a prediction of rapid cognitive decline; and
(c) treating the patient or subject with early and aggressive therapy appropriate to treat AD with rapid cognitive decline.

2. A method of treating a patient with AD or a subject susceptible to developing AD, comprising:
(a) assaying a sample obtained from an early-stage AD patient or a subject susceptible to developing AD for the presence of a BDNF gene mutation;
(b) determining whether the patient or subject is positive for brain Aβ, wherein the presence of brain Aβ in combination with the BDNF gene mutation correlates with a prediction of rapid cognitive decline; and
(c) instructing a healthcare provider to administer early and aggressive therapy appropriate to treat AD with rapid cognitive decline.

3. A method of treating a patient with AD or a subject susceptible to developing AD, comprising:
(a) obtaining a sample from an early-stage AD patient or a subject susceptible to developing AD, and submitting the sample for determination of the presence of a BDNF gene mutation;
(b) ordering a test to determine whether the patient or subject is positive for brain Aβ, wherein the presence of brain Aβ in combination with the BDNF gene mutation correlates with a prediction of rapid cognitive decline; and
(c) treating the patient or subject with early and aggressive therapy appropriate to treat AD with rapid cognitive decline.

4. A method of predicting the rate of cognitive decline expected in a patient with AD or a subject susceptible to developing AD, comprising:
(a) assaying a sample obtained from an early-stage AD patient or a subject susceptible to developing AD for the presence of a BDNF gene mutation; and
(b) determining whether the patient or subject is positive for brain Aβ;
wherein the presence of brain Aβ in combination with the BDNF gene mutation correlates with a prediction of rapid cognitive decline, and indicates a need for rapid, aggressive AD treatment.

5. A method of predicting the rate of cognitive decline expected in a patient with AD or a subject susceptible to developing AD, comprising:
(a) obtaining a sample from an early-stage AD patient or a subject susceptible to developing AD, and submitting the sample for determination of the presence of a BDNF gene mutation; and
(b) ordering a test to determine whether the patient or subject is positive for brain Aβ;
wherein the presence of brain Aβ in combination with the BDNF gene mutation correlates with a prediction of rapid cognitive decline, and indicates a need for rapid, aggressive AD treatment.

6. A method of prognosing a patient with AD or a subject susceptible to developing AD, comprising:
(a) assaying a sample obtained from an early-stage AD patient or a subject susceptible to developing AD for the presence of a BDNF gene mutation; and
(b) determining whether the patient or subject is positive for brain Aβ; wherein the presence of brain Aβ in combination with the BDNF gene mutation correlates with a prediction of rapid cognitive decline, and indicates a need for rapid, aggressive AD treatment.

7. A method of treating a patient with Alzheimer's disease (AD) or a subject susceptible to developing AD, comprising:
(a) assaying a sample obtained from an early-stage AD patient or a subject susceptible to developing AD for the presence of a protein tyrosine phosphatase receptor-type, Z polypeptide 1 (Ptprz1) gene mutation;
(b) determining whether the patient or subject is positive for brain amyloid-beta (Aβ), wherein the presence of brain Aβ in combination with the Ptprz1gene mutation correlates with a prediction of rapid cognitive decline; and
(c) treating the patient or subject with early and aggressive therapy appropriate to treat AD with rapid cognitive decline.

8. A method of treating a patient with AD or a subject susceptible to developing AD, comprising:
(a) assaying a sample obtained from an early-stage AD patient or a subject susceptible to developing AD for the presence of a Ptprz1 gene mutation;
(b) determining whether the patient or subject is positive for brain Aβ, wherein the presence of brain Aβ in combination with the Ptprz1gene mutation correlates with a prediction of rapid cognitive decline; and
(c) instructing a healthcare provider to administer early and aggressive therapy appropriate to treat AD with rapid cognitive decline.

9. A method of treating a patient with AD or a subject susceptible to developing AD, comprising:
(a) obtaining a sample from an early-stage AD patient or a subject susceptible to developing AD, and submitting the sample for determination of the presence of a Ptprz1 gene mutation;
(b) ordering a test to determine whether the patient or subject is positive for brain Aβ, wherein the presence of brain Aβ in combination with the Ptprz1 gene mutation correlates with a prediction of rapid cognitive decline; and
(c) treating the patient or subject with early and aggressive therapy appropriate to treat AD with rapid cognitive decline.

10. A method of predicting the rate of cognitive decline expected in a patient with AD or a subject susceptible to developing AD, comprising:
(a) assaying a sample obtained from an early-stage AD patient or a subject susceptible to developing AD for the presence of a Ptprz1 gene mutation; and
(b) determining whether the patient or subject is positive for brain Aβ; wherein the presence of brain Aβ in combination with the Ptprz1 gene mutation correlates with a prediction of rapid cognitive decline, and indicates a need for rapid, aggressive AD treatment.

11. A method of predicting the rate of cognitive decline expected in a patient with AD or a subject susceptible to developing AD, comprising:
(a) obtaining a sample from an early-stage AD patient or a subject susceptible to developing AD, and submitting the sample for determination of the presence of a Ptprz1 gene mutation; and
(b) ordering a test to determine whether the patient or subject is positive for brain Aβ;
wherein the presence of brain Aβ in combination with the Ptprz1 gene mutation correlates with a prediction of rapid cognitive decline, and indicates a need for rapid, aggressive AD treatment.

12. A method of prognosing a patient with AD or a subject susceptible to developing AD, comprising:
(a) assaying a sample obtained from an early-stage AD patient or a subject susceptible to developing AD for the presence of a Ptprz1 gene mutation; and
(b) determining whether the patient or subject is positive for brain Aβ; wherein the presence of brain Aβ in combination with the Ptprz1 gene mutation correlates with a prediction of rapid cognitive decline, and indicates a need for rapid, aggressive AD treatment.

13. The method of any one of claims 1 to 12, wherein the presence of brain Aβ in combination with the BDNF gene mutation further correlates with a prediction of decline in brain glucose metabolism, as measured by [¹⁸F]-fluorodeoxyglucose positron emission tomography (FDG-PET).

14. The method of any one of claims 1 to 13, wherein brain Aβ is measured by pittsburgh compound B positron emission tomography PiB-PET or [¹⁸F]-AV-45 (florbetapir)-PET.

15. The method of any one of claims 1 to 14, wherein the sample from an early-stage AD patient or a subject susceptible to developing AD comprises fresh, frozen, or preserved tissue, a biopsy, an aspirate, blood or any blood constituent, a bodily fluid, cells, or any combination thereof.

16. The method of any one of claims 1 to 15, wherein the sample is assayed for the presence of the BDNF gene or Ptprz1 gene mutation using a nucleic acid hybridization assay, a nucleic acid polymerization assay, a sequencing assay, or a combination thereof.

17. The method of claim 16, wherein the assay comprises the use of a gene chip array.

18. The method of claim 16 or claim 17, wherein the assay comprises a TaqMan assay, a flap endonuclease assay, genomic DNA sequencing.

19. The method of any one of claims 1 to 18, wherein the presence of the BDNF gene or Ptprz1 gene mutation is determined using a nucleic acid probe specific for the mutation.

20. The method of any one of claims 1 to 19, wherein the BDNF gene and/or Ptprz1 gene mutation comprises a single nucleotide polymorphism (SNP).

21. The method of claim 20, wherein the BDNF gene and/or Ptprz1 gene mutation comprises two or more SNPs.

22. The method of claim 20 or claim 21, wherein the BDNF gene mutation comprises at least one copy of Val66Met (A/G) at rs6265.

23. The method of claim 22, wherein the BDNF gene mutation comprises two copies of Val66Met (A/G) at rs6265.

24. The method of claim 22 or claim 23, wherein a patient positive for both brain Aβ and at least one copy of the Val66Met mutation is predicted to have a faster 36 month cognitive decline than a patient negative for either brain Aβ or a Val66Met mutation.

25. The method of any one of claims 22 to 24, wherein a patient positive for both brain Aβ and at least one copy of the Val66Met mutation is predicted to have a faster decline in brain glucose metabolism than a patient negative for either brain Aβ or a Val66Met mutation.

26. The method of claim 20 or claim 21, wherein the Ptprz1 gene mutation comprises at least one copy of “T” allele at rs6946211.

27. The method of any one of claims 1 to 26, wherein the rate of cognitive decline can be measured by a mini-mental state examination, the clinical dementia rating scale, the Boston name test, a logical memory test, a delayed recall test, or any combination thereof.

28. The method of any one of claims 1 to 27, wherein the therapy comprises administration of an anti-Aβ antibody, or antigen-binding fragment thereof, a cholinesterase inhibitor, an N-methyl-D-aspartate receptor antagonist, or any combination thereof.

29. The method of claim 28, wherein the antibody or fragment thereof is can bind a beta-amyloid plaque, a cerebrovascular amyloid, a diffuse Abeta deposit, a neurofibrillary tangle, or an Abeta protein aggregate; wherein the antibody or its encoding cDNA is derived from B-cells or memory B-cells obtained from a human patient who is symptom-free but affected with or at risk of developing a disorder, or a human patient with an unusually stable disease course, and wherein the antibody has been identified by binding to a specimen of pathologically altered cells or tissue of predetermined clinical characteristics.

30. A method of treating a patient with AD or a subject susceptible to developing AD, comprising administering to the patient or subject an anti-Aβ antibody, or antigen-binding fragment thereof, a cholinesterase inhibitor, an N-methyl-D-aspartate receptor antagonist, or any combination thereof, wherein the patient has (a) at least one mutation in the BDNF gene and/or Ptprz1 gene and (b) brain Aβ.

31. The method of any one of claims 28 to 30, wherein the antibody or fragment thereof comprises a VH and a VL, wherein the VH comprises VHCDR1, VHCDR2, and VHCDR3 amino acid sequences of SEQ ID NOs: 3, 4, 5, and the VL, comprises VLCDR1, VLCDR2, and VLCDR3 amino acid sequences of SEQ ID NOs: 6, 7, 8.

32. The method of any one of claims 28 to 30, wherein the antibody or fragment thereof comprises a VH and a VL, wherein the VH comprises SEQ ID NO: 1 and the VL comprises SEQ ID NO: 2.


SEQUENCE LISTING (8 sequences)

SEQ ID NO: 1 - protein, 124 aa, Artificial Sequence
  VH
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 2 - protein, 108 aa, Artificial Sequence
  VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 3 - protein, 5 aa, Artificial Sequence
  VHCDR1
SYGMH

SEQ ID NO: 4 - protein, 17 aa, Artificial Sequence
  VHCDR2
VIWFDGTKKYYTDSVKG

SEQ ID NO: 5 - protein, 15 aa, Artificial Sequence
  VHCDR3
DRGIGARRGPYYMDV

SEQ ID NO: 6 - protein, 11 aa, Artificial Sequence
  VLCDR1
RASQSISSYLN

SEQ ID NO: 7 - protein, 7 aa, Artificial Sequence
  VLCDR2
AASSLQS

SEQ ID NO: 8 - protein, 9 aa, Artificial Sequence
  VLCDR3
QQSYSTPLT